(19) United States
(12) Patent Application Publication
(10) Pub. No.: US 2025/0134802 A1
(43) Pub. Date: May 1, 2025

(54) Bacteriophages for Protection from Ultraviolet Irradiation

(71) Applicant: THE BOARD OF TRUSTEES OF THE LELAND STANFORD JUNIOR UNIVERSITY, Stanford, CA (US)

(72) Inventors: Maryam HAJFATHALIAN, Mountain View, CA (US); Paul L. BOLLYKY, Palo Alto, CA (US); Michael J. KRATOCHVIL, San Mateo, CA (US)

(73) Assignee: THE BOARD OF TRUSTEES OF THE LELAND STANFORD JUNIOR UNIVERSITY, Stanford, CA (US)

(21) Appl. No.: 18/692,082

(22) PCT Filed: Sep. 16, 2022
(86) PCT No.: PCT/US2022/043894

§ 371 Date: Mar. 14, 2024

Related U.S. Application Data
(60) Provisional application No. 63/245,076, filed on Sep. 16, 2021.

(51) Int. Cl. A61K 8/99; A01N 63/40; A01P 21/00; A61Q 17/04; C12N 7/00

(52) U.S. Cl. CPC A61K 8/99; A01N 63/40; A01P 21/00; A61Q 17/04; C12N 7/00; C12N 2795/00032; C12N 2795/00052

(57) ABSTRACT

Provided herein are ultraviolet light blocking compositions composed of an active agent containing a phage, phage particle, or other functional component thereof, or another viral particle, or other functional component thereof, in addition to one or more secondary agents useful for application to skin, in industrial or domestic settings, or in agriculture.

### CROSS REFERENCE TO RELATED APPLICATIONS

This application claims the benefit of priority of U.S. Provisional Application No. 63/245,076, filed Sep. 16, 2021, entitled “BACTERIOPHAGES FOR PROTECTION FROM ULTRAVIOLET IRRADIATION”, which is incorporated by reference as if fully set forth herein.

### BACKGROUND

Ultraviolet (UV) is a form of electromagnetic radiation with wavelength from 10 nm (with a corresponding frequency around 30 PHz) to 400 nm (750 THz), shorter than that of visible light, but longer than X-rays. UV radiation is present in sunlight. For humans, suntan and sunburn are familiar effects of exposure of the skin to UV light, along with an increased risk of skin cancer.

UV light in the A, B, and C portions of the spectrum is known to be damaging to human tissues and other materials. For example, it is well documented that overexposure to UV-B radiation can cause sunburn and some forms of skin cancer. UV-C is the highest-energy and most-dangerous type of ultraviolet radiation, which is known to cause adverse effects that can variously be mutagenic or carcinogenic. While UV-A has historically been considered to be less harmful than UV-B, it is known today to contribute to skin cancer, e.g., via indirect DNA damage. Some sunscreens contain titanium dioxide, zinc oxide, and avobenzone, which help protect against UV-A rays. However, compounds containing these materials do not degrade easily and tend to persist in the environment. Although there are many sunscreen products available, it has become apparent that common sunscreens require chemicals and active chemical ingredients that have detrimental effects on coral reefs and other aquatic life.

UV rays likewise damage wood, plastic, and other materials leading to discoloration, cracking, loss of strength or disintegration. Also, many pigments in industrial coatings and dyes absorb UV and unfavorably change color. It is known that the effects of UV attack on these materials increases with exposure time and sunlight intensity.

There exists a need in the art for a way to block UV light in a manner that is safe for the environment and the user or material. The present compositions and methods address needs in the art related to these naturally occurring systems and processes.

### SUMMARY

According to presently described embodiments, an ultraviolet light blocking composition is provided comprising an active agent and a secondary agent. Often such active agent is composed of a phage, phage particle, or other functional component thereof, or another viral particle, or other functional component thereof.

Often the bacteriophage is purified from a bacteria cell selected from E. coli or P. aeruginosa. Frequently, the bacteriophage is a Pf phage, T4P phages, Qhix phage, Eb M3 phages, T6 phage, Luz19, and/or Luz24 phage, or a combination of two or more of the foregoing bacteriophages.

Also often the bacteriophage is selected from one or more of the group consisting of Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales,

Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and Spiraviridae.

In frequent embodiments the bacterial cell is modified to produce no endotoxin/lipopolysaccharide or endotoxin/lipopolysaccharide that is not immunogenic to a human subject.

In certain often included embodiments the ultraviolet light blocking composition includes a viral particle as the active agent. Often the viral particle is produced by a mycophage or a eukaryotic cell selected from the group consisting of one or more of: Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobirnaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, and Zobellviridae.

In certain often included embodiments the active agent is a phage-derived nanoparticle. Often in such embodiments the phage-derived nanoparticle is an icosahedron-shaped phage-derived nanoparticle. Frequently the icosahedron-shaped or filamentous phage-derived nanoparticle is comprised of a phage membrane protein, a peptidoglycan, a teichoic acid, a lipid-containing phage envelope, a lipid containing phage shell, or a combination (cocktail) thereof.

According to often included embodiments, the secondary agent comprises one or more of glycerin, a betaines, ethanol, butyloctyl salicylate, an acrylate polymer, polyethylene glycol (PEG), propylene glycol, stearate, arachidoyl alcohol, stearyl alcohol, an acrylate, xanthan gum, disodium ethylenediaminetetraacetic acid (EDTA), a retinoid, an alpha-hydroxy acid such as glycolic acid, lactic, tartaric acid, citric acid, a beta-hydroxy acid, hydroquinone, kojic acid, L-ascorbic acid, vitamin C0, a glycosaminoglycan, a copper peptide, alpha-lipoic acid, dimethylaminoethanol, a hormone, a growth factor, a surfactant, a thickening agent such as hydroxyethylcellulose, xanthan, carrageenan, alginate or chemically modified cellulose compounds, an emulsifier such as isopropyl myristate (IPM) or diisopropyl adipate, or a polyacrylate, an aromatic agent such as anise alcohol, benzyl alcohol, coumarin, eugenol, hydroxycitronellal, limonene or geraniol, a polyhydroxy acid, a preservative, an antioxidant, and/or a pigment. Often the secondary agent comprises a thickening agent, an emulsifier, an aromatic agent, an alpha-hydroxy acid, a polyhydroxy acid, a preservative, an antioxidant, a pigment or a combination of two or more of the foregoing. Frequently the secondary agent comprises or further comprises a retinoid, a Beta-hydroxy acid, Hydroquinone, Kojic acid, L-ascorbic acid, vitamin CO, a glycosaminoglycan, copper, Alpha-lipoic acid, dimethylaminoethanol, a hormone, a growth factor, or a combination of two or more of the foregoing.

According to frequent embodiments, the composition comprises a sunscreen, a paint, an industrial coating, a stain, a lacquer, or a solution for agricultural application to sun-sensitive crops.

In frequent embodiments, the ultraviolet light blocking composition is provided containing an active agent comprising a phage, phage particle, or other functional component thereof, or another viral particle, or other functional component thereof at a concentration of between 10⁵ to 10¹² active agent per mm² when spread in a thin film.

Often, the ultraviolet light blocking provides between 60% to 100% blocking of visible light, UVA, UVB, and/or UVC radiation. Often, the ultraviolet light blocking provides between 70% to 100% blocking of visible light, UVA, UVB, and/or UVC radiation. Often, the ultraviolet light blocking provides between 80% to 100% blocking of visible light, UVA, UVB, and/or UVC radiation. Often, the ultraviolet light blocking provides between 90% to 100% blocking of visible light, UVA, UVB, and/or UVC radiation. Often, the ultraviolet light blocking provides between 60% to 90% blocking of visible light, UVA, UVB, and/or UVC radiation. Often, the ultraviolet light blocking provides between 70% to 90% blocking of visible light, UVA, UVB, and/or UVC radiation. Often, the ultraviolet light blocking provides between 80% to 90% blocking of visible light, UVA, UVB, and/or UVC radiation. Often, the ultraviolet light blocking provides between 60% to 80% blocking of visible light, UVA, UVB, and/or UVC radiation. Often, the ultraviolet light blocking provides between 70% to 80% blocking of visible light, UVA, UVB, and/or UVC radiation. Often, the ultraviolet light blocking provides between 60% to 70% blocking of visible light, UVA, UVB, and/or UVC radiation.

According to other frequent embodiments, a kit for the production of a skin care formulation is provided having the ability to block ultraviolet light, comprising a plurality of bacteriophages in suspension (e.g., between 10⁵ to 10¹² active agent per mm² when spread in a thin film) in a physiologically acceptable carrier, and instructions for combination of the bacteriophage suspension with a skin care formulation.

All patents, patent applications, and publications mentioned herein, both supra and infra, are hereby incorporated by reference in their entireties.

### BRIEF DESCRIPTION OF THE FIGURES

The skilled person in the art will understand that the drawings, described below, are for illustration purposes only. The drawings are incorporated in and constitute a part of this specification.

FIG. 1 depicts UV light spectrum of a SPF50 commercial sunscreen coating applied to a cuvette. The boundaries of UV-A, -B, and -C radiation spectra are designated by shading depicted in the plots.

FIG. 2 depicts the UV light spectrum of a solution of an E.coli phage at approximately 10⁹ PFU/mL. The boundaries of UV-A, -B, and -C radiation spectra are designated by shading depicted in the plots.

FIGS. 3A-F depict the extinction spectra for various phages at different concentrations. FIG. 3A. depicts the extinction spectra for LUZ 24 at approximately 10⁶ PFU/mL. FIG. 3B. depicts the extinction spectra for Luz 19 at approximately 10¹¹ PFU/mL. FIG. 3C. depicts the extinction spectra for E Coli (Eb M3) at approximately 10¹¹ PFU/mL. FIG. 3D. depicts the extinction spectra for Qhix at approximately 10¹² PFU/mL. FIG. 3E depicts the extinction spectra for Pf at approximately 10¹⁰ PFU/mL. FIG. 3F depicts the extinction spectra for T4P at approximately 10⁹ PFU/mL.

FIGS. 4A-4C depict the extinction spectra of Pf and T4P phages. FIG. 4A depicts the extinction spectra of Pf. FIG. 4B depicts the extinction spectra of T4P. FIG. 4C depicts the extinction spectra of a cocktail of Pf and T4P with a broad UV spectrum (with PF at approximately 10¹⁰ PFU/mL, and T4P at approximately 10⁹ PFU/mL).

FIGS. 5A-C provide UV-A stability data and transition electron microscopy (TEM) images for a cocktail of Pf and T4P phages. FIG. 5A depicts the optical extinction spectra of the Pf and T4P cocktail before and after UV-A irradiation for 40 minutes. FIG. 5B and 5C shows the TEM images of the Pf and T4P cocktail before UV-A irradiation for 40 minutes. FIG. 5C shows the TEM images of the Pf and T4P cocktail after UV-A irradiation for 40 minutes.

FIG. 6A provides extinction spectra data of the phage LUZ24 and UV light absorption at different concentrations.

FIG. 6B provides extinction spectra data of the phage Pf and UV light absorption at different concentrations.

FIG. 7A provides data showing the sizes of T-Series E. coli phages originally produced in May 1972 and stored in air-tight containers until analysis in June 2021. FIG. 7B consists of Table 1. Table 1 depicts the initial 1972 phage titers (i.e., active concentration) of the phages (of FIG. 5A) at approximately 10⁹ plague forming units at the time of storage and titers of the 1972 phages after 48 years of storage.

FIG. 8 provides data showing the infectivity loss of selected phages under UV-C light irradiation for 20 minutes.

FIG. 9A and 9B depict the optical extinction spectra of commercial skin care products with and without cocktail phages (Pf+T4P).

### DETAILED DESCRIPTION

For clarity of disclosure, and not by way of limitation, the detailed description of the various embodiments is provided into certain subsections that follow. A single embodiment may be discussed in multiple subsections.

Unless otherwise defined herein, scientific and technical terms used in connection with the present application shall have the meanings that are commonly understood by those of ordinary skill in the art to which this disclosure belongs. This disclosure is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeck, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein “composition” and “formulation” may be used interchangeably, each composed of two or more ingredients, including an active agent.

As used herein, an “active agent” refers to a phage, phage particle, or other functional component thereof, or another viral particle, or other functional component thereof, each of which blocks or absorbs visible light and/or UV radiation.

Medical organizations recommend that patients protect themselves from UV radiation by using sunscreen. Damage from natural UV irradiation can lead to both acute (in the form of sun burns) and chronic skin damage (in the form of accelerated aging of the skin, discoloration) as well as dangerous skin cancers. Sunscreen reduces the direct DNA damage that causes sunburn, by blocking UV-B, and the usual SPF rating indicates how effectively this radiation is blocked. SPF is, therefore, also called UVB-PF, for “UV-B protection factor”. This rating, however, offers no data about important protection against UVA, which does not primarily cause sunburn but is still harmful, since it causes indirect DNA damage and is also considered carcinogenic. Protection from harmful ultraviolet radiation produced by the sun, therefore, is critical in a variety of settings, including to human health in the form of skin protection.

Current UV-protection products such as sunscreens rely on both organic and inorganic ingredients. These include organic chemical absorbers or blockers that absorb UVA/UVB rays, such as avobenzone, oxybenzone and octyl methoxycinnamate. They can also include inorganic absorbers/blockers of UV radiation such as carbon black, titanium dioxide, and zinc oxide. UV stabilizers in plastics usually act by absorbing the UV radiation preferentially and dissipating the energy as low-level heat. Active agents currently relied upon in both sunscreens and industrial applications such as paints, plastics, fabrics, and protective coatings are highly susceptible to degradation and comprised of non-natural chemicals. Therefore, the present inventors have recognized that replacement of these artificial and often potentially environmentally damaging UV absorbing materials with more environmentally and user-friendly materials would provide profound benefits.

As described herein, naturally occurring and/or engineered bacteriophages (phages) delivered within topical creams, lotions, lip balms and other settings as novel class of UV-absorbing materials are provided. Further, according to the present disclosure, naturally occurring and/or engineered bacteriophages are incorporated into industrial materials such as paints, plastics, fabrics, and thin protective coatings. It has been discovered by the inventors that phages alone or together with additional core ingredients of topical creams, lotions, lip balms, paints, plastics, fabrics, and/or thin protective coatings specified are useful as UV blockers in these compositions for human and industrial usage.

As presented herein, the present disclosure provides bacteriophages (phages) as novel class of UV-absorbing particles. Phages are naturally occurring virus that target bacteria with extreme specificity. Phages are comprised of a nucleic acid surrounded by a protein structure. Generally speaking, this protein structure is comprised of a head and a tail. The head has an icosahedron shape that contains the nucleic acid used to infect bacteria. They have a structure that has evolved to be highly specific and effective at hijacking the cellular machinery of a host bacteria cell to produce copies of itself. As such, phages can be produced by bacteria previously infected with a phage, and thus can be produced efficiently using bacterial strains. The present inventors have understood that phages are naturally produced with great structural precision and in large number using bacterial expression. Therefore, in a short period of time, very large concentrations of chemically and structurally identical phages (>10¹⁰/mL of expression) can be naturally produced. As such natural source products are produced in large quantity and with very high precision and quality control. Furthermore, techniques for producing a non-infective Escherichia coli bacteriophage T4 without viral DNA in the final virus are known and incorporated herein. According to embodiments contemplated herein a E. coli phage may be modified to render it incapable of infecting a bacterial cell.

It has been discovered by the present inventors that a plurality of lytic phages are capable of absorbing UV irradiation in both liquid phase or when deposited as a thin film, e.g., between 10⁵ to 10¹² phage particles per mm². Based on this, it has been determined that such phages can be incorporated into a sunscreen lotion and provide protection from UV irradiation similar to, and often surpassing, conventional sunscreens.

According to frequently included embodiments described herein a UV blocking formulation is provided containing a Pf phage, Eb M3 phage, T4P phage, T6 phage, Qhix phage, Luz19 phage, or Luz24 phage, or a combination (cocktail) of two or more of the foregoing. Such formulation is often a sunscreen, a paint, an industrial coating, a stain, a lacquer, or a solution for agricultural application to sun-sensitive crops. Often such formulations are provided such that when applied as a thin film, they provide between 10⁵ to 10¹² phages per mm².

Phages are viruses produced by bacteria. Phages are abundant within the human body and within the environment. It is estimated that over 10¹⁶ phages are present within each of us, outnumbering both our bacteria as well as our own cells. Most of these phages are present at sites of bacterial colonization, including the intestine, urogenital tract and lungs. Despite great morphologic structural heterogeneity and genetic diversity, phages share certain common features that may be relevant to immune recognition. Further phages are typically non-enveloped viruses, as the bacteria that produce them lack eukaryotic cell membranes. The majority characterized to date have DNA genomes (while RNA viruses are present in the gut, these are thought to be derived from ingested plant matter).

As described herein, naturally occurring, and engineered bacteriophages are incorporated in creams, lotions, lip balms and other topical preparations. Further, as contemplated herein, naturally occurring and/or engineered bacteriophages are incorporated into industrial materials such as paints, plastics, fabrics, and thin protective coatings. The present disclosure presents a unique novel union of phages with these existing products and materials for UV protection.

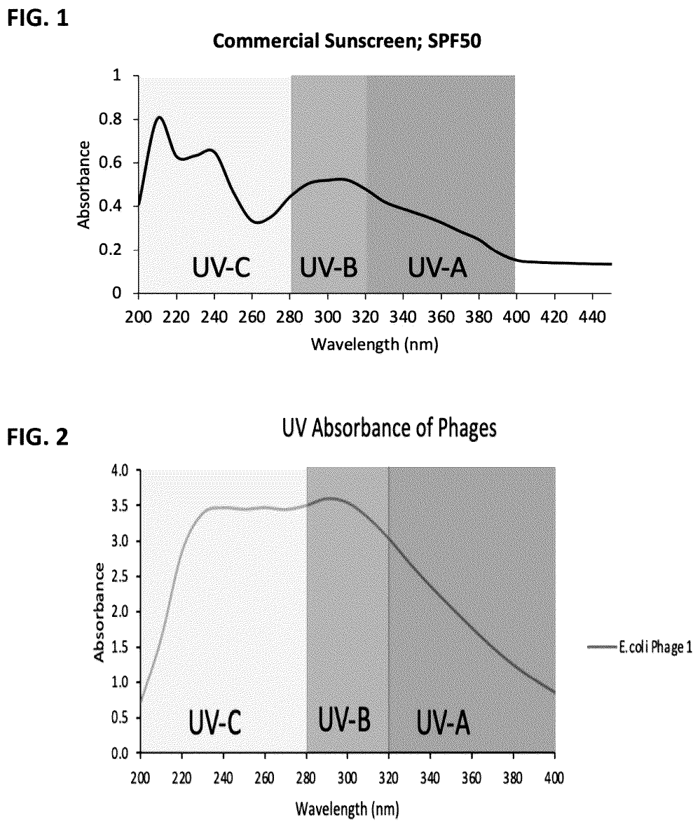

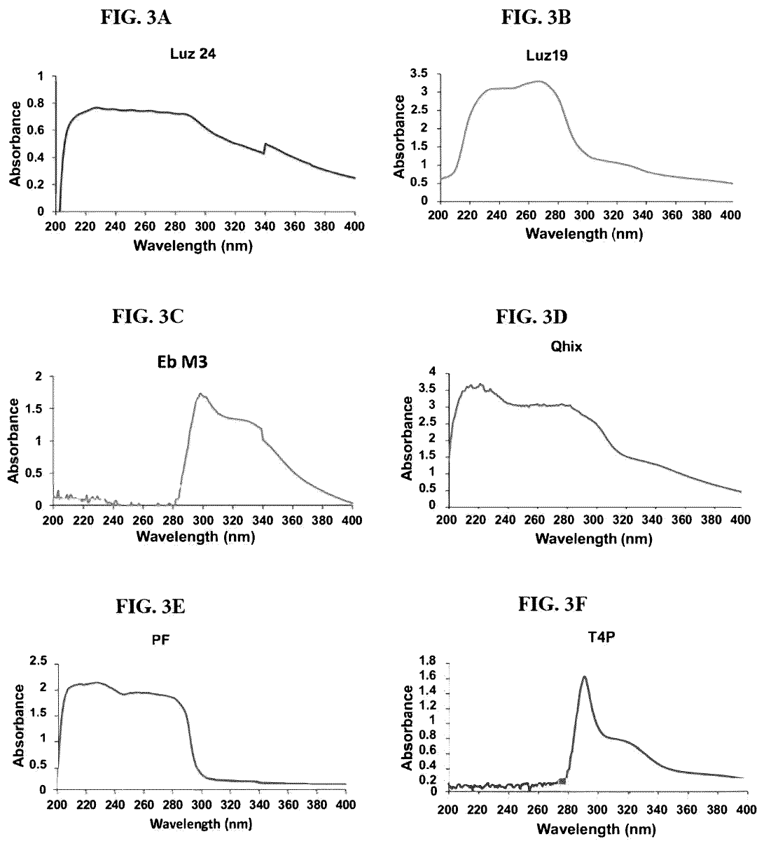

As presented herein, a set of lytic phages have been identified that are capable of absorbing UV irradiation in both liquid phase or when deposited as a thin film (FIGS. 2-3). Compared with commercially available sunscreen of SPF50, the UV-A, UV-B, and UV-C (FIG. 1) absorbance is at least equivalent to the commercial sunscreen or have much greater coverage in certain UV spectra.

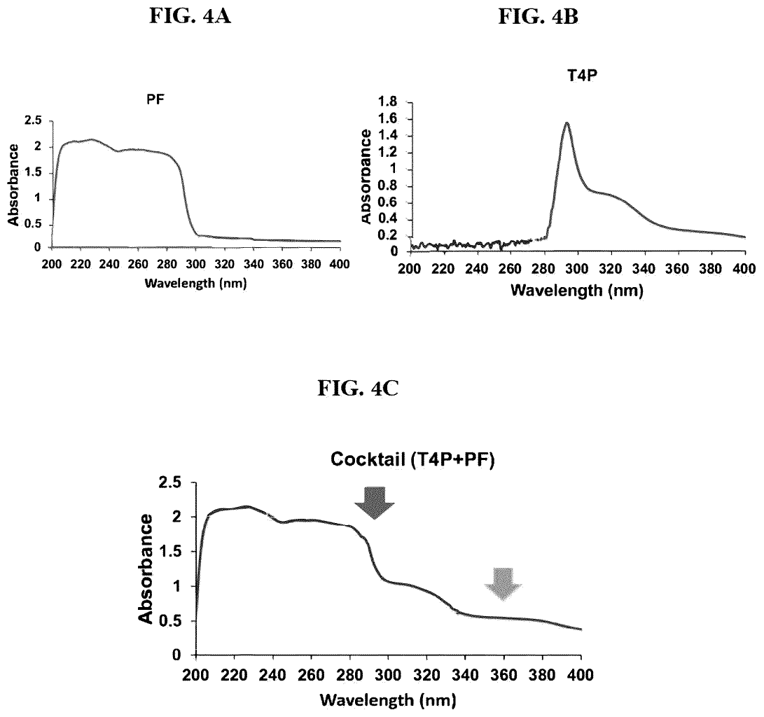

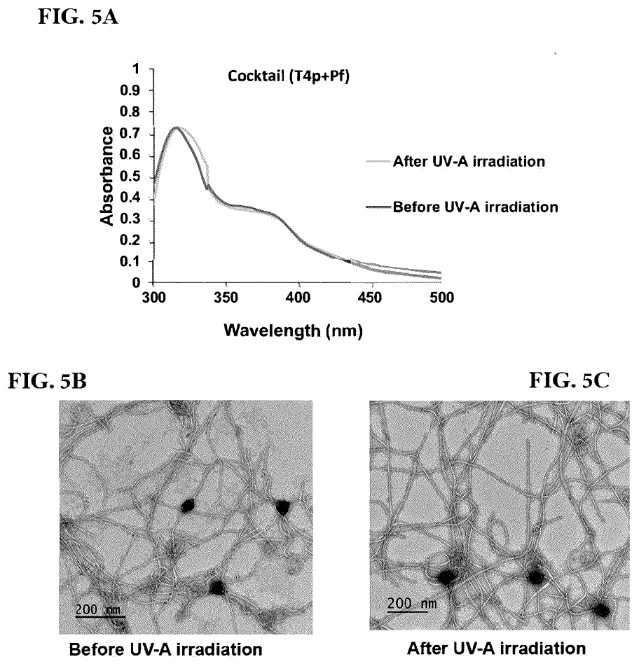

A variety of phages and/or a cocktail of T4P and Pf phages (FIGS. 3 and 4 A-C) have been examined by the present inventors for UV absorption abilities and similar properties by the present inventors. The inventors have discovered that the phages of the presently contemplated embodiments are stable under UV light. The phage cocktail is shown to provide a complete coverage in whole UV spectrum and remain stable during and after exposure to the UV light for 40 minutes. (FIGS. 5A-C). Optical extension spectra and TEM images for the phage cocktail that imparts UV protection are also presented herein, defining exemplary embodiments of phages, including combinations and mixtures (cocktails) thereof, that provide such operability. Cocktails of two or more of LUZ 19, LUZ24, EB M3, Qhix, Pf and/or T4P are specifically contemplated in frequent embodiments provided herein.

According to the presently contemplated embodiments, an ultraviolet light blocking composition is provided that is capable of blocking UV radiation, which composition is incorporated in formulations for mammal use, industrial use, domestic use, or agricultural use.

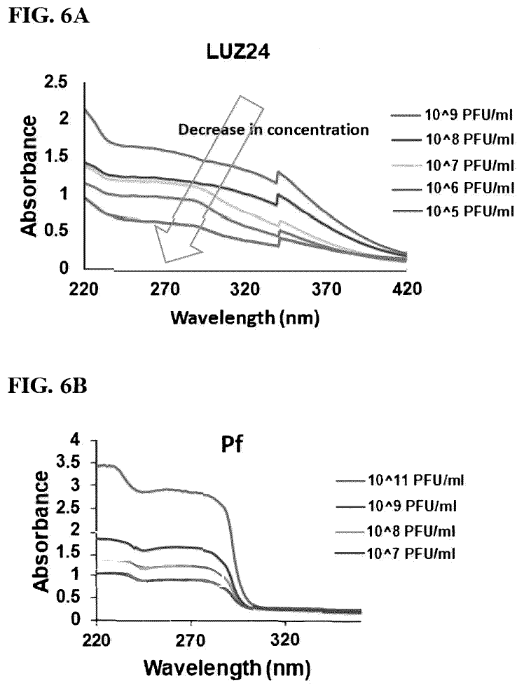

In certain embodiments, the resulting formulation is provided such that when applied it forms a film or coating comprised of 10⁵ to 10¹² phage particles per mm². Such film provides the described aspects of UV blocking in the contemplated formulations. In the contemplated embodiments, a coating providing 10⁵ to 10¹¹ phage particles per mm² provides up to 100% blocking of UVA, up to 100% blocking of UVB, and/or up to 100% blocking of UVC radiation (FIG. 6A and B). In certain embodiments, a coating providing 10⁵ to 10⁷ or 10⁵ to 10¹² phage particles per mm² provides up to 90% blocking of UVA, up to 90% blocking of UVB, and/or up to 90% blocking of UVC radiation. In other embodiments, a coating providing 10⁵ to 10⁷ or 10⁵ to 10¹² phage particles per mm² provides up to 80% blocking of UVA, up to 80% blocking of UVB, and/or up to 80% blocking of UVC radiation. In other embodiments, a coating providing 10⁵ to 10⁷ or 10⁵ to 10¹² phage particles per mm² provides up to 70% blocking of UVA, up to 70% blocking of UVB, and/or up to 70% blocking of UVC radiation. In other embodiments, a coating providing 10⁵ to 10⁷ or 10⁵ to 10¹² phage particles per mm² provides up to 60% blocking of UVA, up to 60% blocking of UVB, and/or up to 60% blocking of UVC radiation. In certain other embodiments, the blocking of UV spectra varies across the different spectra in the percentages noted.

In certain embodiments, a coating providing 10⁵ to 10⁷ or 10⁵ to 10¹² phage particles per mm² provides up to 100% blocking of visible light. In certain embodiments, a coating providing 10⁵ to 10⁷ or 10⁵ to 10¹² phage particles per mm² provides up to 90% blocking of visible light. In certain embodiments, a coating providing 10⁵ to 10⁷ or 10⁵ to 10¹² phage particles per mm² provides up to 80% blocking of visible light. In certain embodiments, a coating providing 10⁵ to 10⁷ or 10⁵ to 10¹² phage particles per mm² provides up to 70% blocking of visible light. In certain embodiments, a coating providing 10⁵ to 10⁷ or 10⁵ to 10¹² phage particles per mm² provides up to 60% blocking of visible light.

In certain embodiments, the concentration of phages in the coating provides between 10⁵ to 10⁹ or 10⁵ to 10¹² phage particles per mm². In such embodiments, the percentage of visible light and UV radiation blocked is within the percentages noted above, which description is incorporated herein by reference. In general, the larger the concentration of phages per mm² in the coating, the larger amount of visible light and/or UV spectra that is blocked.

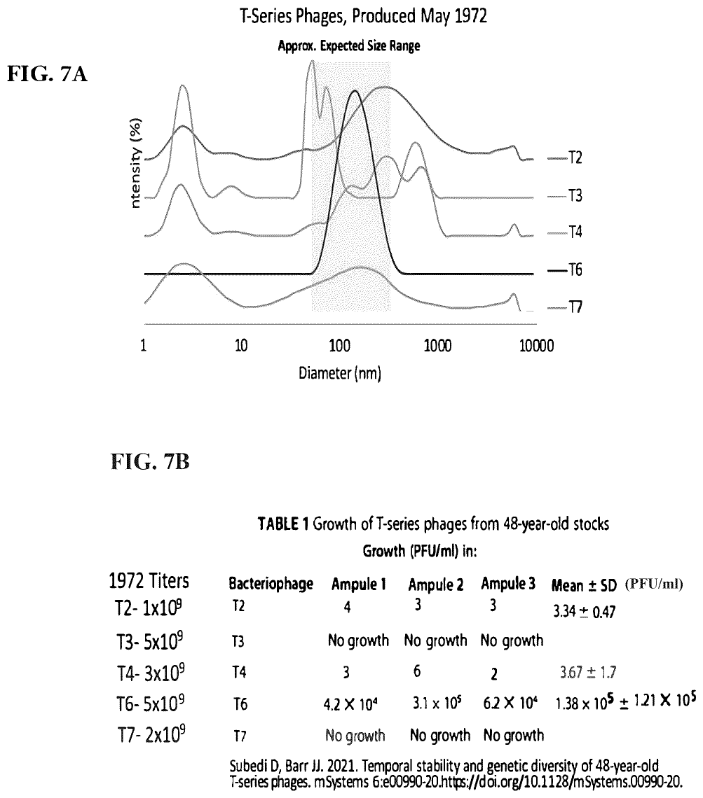

Furthermore, the presently contemplated embodiments comprise phages that are stable over time, under typical environmental conditions, and/or at room temperature or above, and also after prolonged exposure to sunlight. For example, the present inventors have identified and grown phages contemplated in the present embodiments after prolonged storage, which phages demonstrated robust stability over time (FIGS. 7A & 7B). The T6 phage is shown, for example, to be approximately the correct size after removal from long term storage, suggesting a high relative stability of the phage from degradative or aggregative processes that could disrupt the phage's UV absorbance ability.

It is contemplated that the presently described formulation and composition embodiments provide a stable active agent (i.e., phage or viral particle) after prolonged exposure to sunlight. It is also contemplated that the presently described formulation and composition embodiments provide a stable active agent (i.e., phage or viral particle) after prolonged exposure to temperatures at or above ambient or room temperatures (i.e., at or about 68° F. to 77° F.). It is also further contemplated that the presently described formulation and composition embodiments provide a stable active agent (i.e., phage or viral particle) after prolonged exposure to oxygen or air.

Given the range and diversity of bacteriophages that protect against UV light (including phages from both Gram positive and Gram-negative bacteria), it is herein presented that the ability to deflect UV light is a generalized feature of many viral particles that are exposed to sunlight as part of their life cycle. Thus, many different bacteriophages are contemplated as suitable for the present compositions and uses in preparations intended to block UV light. This set of phages includes both enveloped and non-enveloped phages and phages with DNA and RNA genomes. This set of phages includes phages from at least the following taxonomic groups: Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales, Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and/or Spiraviridae. Combinations (cocktails) of two or more of these taxonomic groups are also contemplated.

Moreover, viral particles made by fungi (mycophages) or eukaryotic cells (viruses) are also contemplated as suitable for the presently contemplated compositions and uses. These include at least the following taxonomic groups: Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guclinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobirnaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, and/or Zobellviridae. Combinations of two or more of these taxonomic groups are also contemplated.

Further combinations (cocktails) of one or more pure or mixed viral particles made by fungi (mycophages) or eukaryotic cells (viruses) and one or more pure or mixed phages produced by bacteria are also contemplated.

Furthermore, as noted, modified phages are useful in the presently contemplated compositions and methods. Such modified phages include, for example, phages stripped of bacterial endotoxin/lipopolysaccharide. Endotoxin/lipopolysaccharide is associated with products produced from bacteria but known to have adverse biological effects on mammals. Thus, for some applications (e.g., topical skin applications), care needs to be taken to eliminate or limit contaminating endotoxin/lipopolysaccharide. Providing for these purified compositions lacking bacterial endotoxin/lipopolysaccharide is contemplated to include via the production of phages using ClearColi E. coli strains (commercially available E. coli strains with genetically modified LPS that does not trigger an immune response in mammalian cells) and other bacterial strains that produce phages yet do not produce lipopolysaccharide or produce versions of lipopolysaccharide that avoid triggering an immune response. Alternatively, or in addition, phages are purified using polymyxin B columns (polymyxin binds endotoxins in solution), through the use of Cesium chloride purification, or other purification methods to remove endotoxin/lipopolysaccharide.

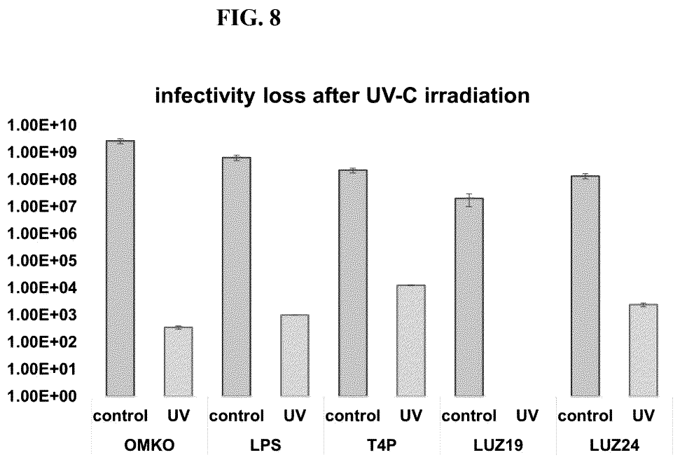

Additional phage modifications contemplated herein include the elimination of the infectious potential of phages. This can be done, for example, via UV-C irradiation (FIG. 8), sonication (physically damaging the phages rendering the phages incapable of “docking” and infecting on bacteria), chemical fixation (addition of oxidizing agents or other chemicals that irreparably damage the genetic material necessary for infection or agents that damage the protein machinery necessary for infection such as formaldehyde, etc.), genetic modification (genetically modifying the phage producing bacteria strains to be without necessary machinery for reproduction, such as removal of the adenosine triphosphatase (ATPase) enzymes necessary for packaging the genetic material within the phages), and/or other means.

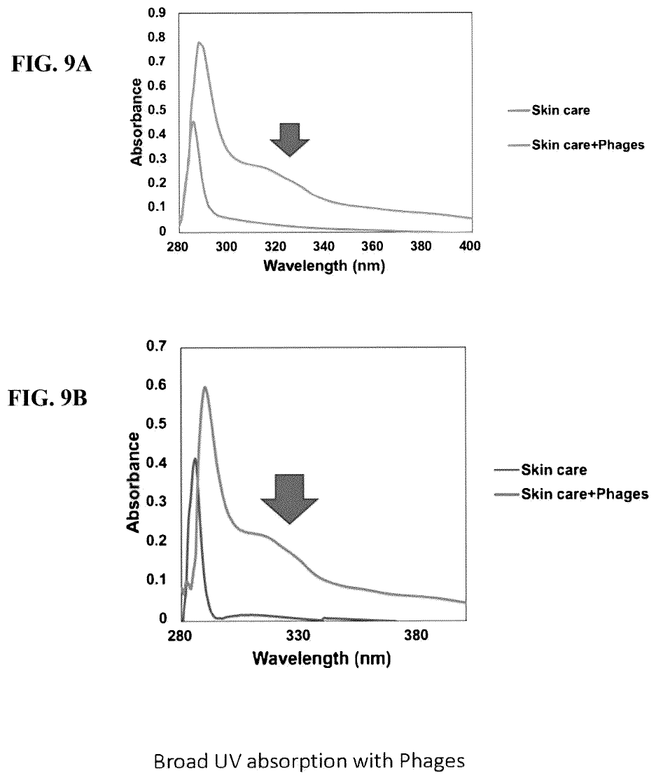

Moreover, the present embodiments can be added to current commercial skin care products such as hand moisturizing, ointments, make up removers, lotions, and hand sanitizers to safely protect UV absorption while keeping its effectiveness (FIGS. 9A and 9B). For example, having a new formulation of hand sanitizers plus phages might give the great UV protection for derivers to use dual function sanitizers.

In certain embodiments, the phages for use in the presently described formulations are modified to block encapsulation of nucleic acid thus producing “dummy” phages with no means of infecting or replicating further. And the combination of the above-described approaches, an engineered endotoxin-free, non-infective, pro-skin regeneration phage is produced that provides protection from UV rays without the associated environmental or human health risks of popular and conventional UV absorption active agents.

Further contemplated modifications include the production and use of phages that provide other enhancing qualities or agents in addition to blocking UV radiation. For example, in certain embodiments, phage capsid proteins are engineered to provide or deliver retinoids (e.g., retin-a, etc.), Beta-hydroxy acid (salicylic acid), Hydroquinone, Kojic acid, L-ascorbic acid (vitamin CO, glycosaminoglycans (e.g. hyaluronan, etc.), copper peptide, Alpha-lipoic acid, DMAE (dimethylaminoethanol), hormones, and/or growth factors. One or more of these enhancing agents are often included in sunscreen embodiments of the present disclosure. In certain embodiments, therefore, phages are modified using phage-display to display active agents. For example, in certain embodiments, phages are modified using phage-display to display retinoids, Beta-hydroxy acid (salicylic acid), Hydroquinone, Kojic acid, L-ascorbic acid (vitamin C0, glycosaminoglycans (e.g. hyaluronan, etc.), copper peptide, Alpha-lipoic acid, DMAE (dimethylaminoethanol), hormones, and/or growth factors.

The present embodiments are often prepared, for example, with thickening agents (e.g. xanthan, carrageenan, alginate or chemically modified cellulose compounds), emulsifiers (e.g. isopropyl myristate (IPM) or diisopropyl adipate, polyacrylates, etc.), aromatic agents (e.g. anise alcohol, benzyl alcohol, coumarin, eugenol, hydroxycitronella, limonene and geraniol), alpha-hydroxy acids (glycolic, lactic, tartaric, citric acids, etc.), Polyhydroxy acids, preservatives, antioxidants, pigments, or and other additives included in skin creams, ointments, gels, cosmetics, and/or hair products, etc. to produce sunscreens of the present disclosure. Such sunscreens are healthy for users and also comprise ingredients that are not harmful to the environment in the concentrations used in such sunscreens. Such sunscreens may be comprised in lotions, ointments, creams, sprays, gels, lip balms, cosmetics, hair products, and/or other preparations intended for topical use. Concentrating agents may also be incorporated in the contemplated formulations to concentrate and organize phages by binding the phages to the polymer backbone to improve the effectiveness of the application and protection of the UV absorptive phages.

These preparations are contemplated to include phages delivered together with, for example, water, glycerin, betaines, Ethanol, butyloctyl salicylate, acrylate polymers, polyethylene glycol (PEG), Propylene glycol, stearate, arachidoyl alcohol, stearyl alcohol, acrylates, xanthan gum, disodium ethylenediaminetetraacetic acid (EDTA), retinoids (e.g. retin-a, etc.), Beta-hydroxy acid (e.g. salicylic acid), Hydroquinone, Kojic acid, L-ascorbic acid (vitamin C0, glycosaminoglycans (e.g. hyaluronan, etc.), copper peptide, Alpha-lipoic acid, and/or DMAE (dimethylaminoethanol). Hormones, growth factors, surfactants (e.g. Polysorbate 20 (Tween 20), thickening agents (e.g. Hydroxyethylcellulose, xanthan, carrageenan, alginate or chemically modified cellulose compounds), emulsifiers (e.g. isopropyl myristate (IPM) or diisopropyl adipate, polyacrylates, etc.), aromatic agents (e.g. anise alcohol, benzyl alcohol, coumarin, eugenol, hydroxycitronellal, limonene and geraniol), alpha-hydroxy acids (glycolic, lactic, tartaric, citric acids, etc.), Polyhydroxy acids, preservatives, antioxidants, pigments, or and other additives may also be optionally incorporated in the compositions contemplated herein.

Other embodiments are contemplated to include UV absorbing phages as an active ingredient in existing formulations of paints, coatings, stains, and/or lacquers. In this regard, such phages may be operably combined with polymers, epoxies, silicone, polyurethanes, acrylics, inks, and/or paints.

Further, other embodiments are contemplated to include UV absorbing phages in agricultural sprays and protection for sun-sensitive crops. Such phages are contemplated as included in preparations formulated with water, PEG glycol stearate, and/or glycerin.

According to contemplated embodiments, a formulation is provided containing between 10⁵ to 10⁹ phage particles per mm² when spread in a thin film. In addition, such formulations often contain one or more of water, glycerin, betaines, Ethanol, butyloctyl salicylate, acrylate polymers, polyethylene glycol (PEG), Propylene glycol, stearate, arachidoyl alcohol, stearyl alcohol, acrylates, xanthan gum, disodium ethylenediaminetetraacetic acid (EDTA), retinoids (e.g. retin-a, etc.), Beta-hydroxy acid (e.g. salicylic acid), Hydroquinone, Kojic acid, L-ascorbic acid (vitamin C0, glycosaminoglycans (e.g. hyaluronan, etc.), copper peptide, Alpha-lipoic acid, and/or DMAE (dimethylaminoethanol). Hormones, growth factors, surfactants (e.g. Polysorbate 20 (Tween 20), thickening agents (e.g. Hydroxyethylcellulose, xanthan, carrageenan, alginate or chemically modified cellulose compounds), emulsifiers (e.g. isopropyl myristate (IPM) or diisopropyl adipate, polyacrylates, etc.), aromatic agents (e.g. anise alcohol, benzyl alcohol, coumarin, eugenol, hydroxycitronellal, limonene and geraniol), alpha-hydroxy acids (glycolic, lactic, tartaric, citric acids, etc.), Polyhydroxy acids, preservatives, antioxidants, pigments, or and other additives. Such pahes often fall into one or more of the following taxonomic groups: Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales, Sphacrolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and/or Spiraviridae. In certain embodiments, the phages in the formulation are modified to provide beneficial proteins and/or modified or purified to remove harmful aspects such as endotoxin and to block the incorporation of nucleic acid. In related embodiments, the phages may be modified to be limited to portions of the phage such as the head.

In certain embodiments, there is provided a method to reduce or eliminate impact of ultraviolet light for a mammal or an inanimate object in the presence of the ultraviolet light. A surface, which may be a skin surface of a mammal or a surface of an inanimate object, may be contacted with a composition comprising an active agent, which may be a bacteriophage, a viral particle, or a phage-derived nanoparticle. The method comprises providing a composition comprising a bacteriophage, a viral particle, or a phage-derived nanoparticle with a secondary agent. Other agents may be provided in this composition. The composition may be applied to the surface by rubbing, spraying, spreading, painting using a brush, or otherwise contacting the composition with the surface to which it is applied. The surface may be the skin of an animal such as a mammal, including a human. The surface may also be an inanimate object, such as a building wall, roof, or other building or structure part, a furniture surface, a structural cable, floors and other ground surfaces, eyeglasses, or glass walls and/or windows on buildings or vehicles.

Also in certain embodiments, including related embodiments, a composition is formulated as a sunscreen comprising an active agent such as a bacteriophage, a viral particle, or a phage-derived nanoparticle. A method to limit or eliminate ultraviolet light impact on skin surfaces of mammals comprises providing a mammal in the presence of ultraviolet light, such as sunlight, with this sunscreen and rubbing this sunscreen on the mammal's skin surface to reduce or eliminate ultraviolet light impact.

As provided in the presently contemplated embodiments, the compositions provided herein are formulated as or within a paint, a stain, a lacquer, or an industrial coating comprising an active agent such as a bacteriophage, a viral particle, or a phage-derived nanoparticle. A method to limit or eliminate ultraviolet light impact on surfaces of inanimate objects comprises coating an inanimate object, such as sunlight, with this paint, stain, lacquer, or industrial coating on the object's surface to reduce or eliminate ultraviolet light impact when the object is exposed to ultraviolet light. Such inanimate objects include, but are not limited to, walls, furniture, vehicles, electronics, sunglasses, and glass walls. The composition may be manufactured as a paint-like solution capable of being applied to a surface using a brush, rollers, submersion, or the like. The composition may also be manufactured as a spray solution capable of being applied to a surface using a spray can or the like.

In further embodiments, a composition is formulated as an agricultural application solution comprising an active agent such as a bacteriophage, a viral particle, or a phage-derived nanoparticle. A method to limit or eliminate ultraviolet light impact on sun-sensitive crops comprises spraying the sun-sensitive crops in the presence of ultraviolet light, such as sunlight, with agricultural application solution to reduce or eliminate ultraviolet light impact. In such embodiments, the composition may be formulated containing or as part of a nutritive and/or pest-reducing composition known in the agricultural arts that is routinely or occasionally provided to such crops during their growth cycle.

In certain embodiments described herein, a method to manufacture a composition for spreading onto the skin of a mammal to reduce or eliminate ultraviolet light impact is provided, the method comprises combining an active agent, the active agent being bacteriophage, a viral particle, or a phage-derived nanoparticle with a secondary agent and/or other agents suitable for manufacturing a composition for spreading, onto the skin of a mammal. The composition may be a sunscreen.

As contemplated in certain embodiments described herein, a method to manufacture a composition for painting or spraying onto an inanimate object is provided, the method comprises combining an active agent, the active agent being bacteriophage, a viral particle, or phage-derived nanoparticle, with a secondary agent and/or other agents suitable for manufacturing a composition for painting or spraying onto an inanimate object. The composition may be a paint, a stain, a lacquer, or an industrial coating.

In further embodiments, a method to manufacture a composition for spraying onto agricultural crops is provided, the method comprises combining an active agent, the active agent being bacteriophage, a viral particle, or a phage-derived nanoparticle, with a secondary agent and/or other agents suitable for manufacturing a composition for spraying onto sun-sensitive agricultural crops. The composition may be manufactured containing or as part of a nutritive and/or pest-reducing composition known in the agricultural arts that is routinely or occasionally provided to such crops during their growth cycle.

The above examples and embodiments are included for illustrative purposes only and are not intended to limit the scope of the disclosure. Many variations to those methods, systems, kits, and devices described above are possible. Since modifications and variations to the examples described above will be apparent to those of skill in this art, it is intended that this invention be limited only by the scope of the appended claims. All publications and references cited herein are expressly incorporated herein by reference in their entirety and/or for the specific reason for which they are cited herein.

1. An ultraviolet light blocking composition comprising an active agent and a secondary agent, wherein the active agent comprises a bacteriophage, a viral particle, or a phage-derived nanoparticle.

2. The ultraviolet light blocking composition of claim 1, wherein, if present, the bacteriophage is selected from the group consisting of Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales, Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and Spiraviridae;
wherein, if present, the viral article is produced by a mycophage or a eukaryotic cell selected from the group consisting of one or more of: Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridae, Mesoniviridae, Metaviridae, Maxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobirnaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, and Zobellviridae,
wherein, if present, the phage-derived nanoparticle is an icosahedron-shaped or filamentous phage-derived nanoparticle, and
wherein the secondary agent comprises one or more of glycerin, a betaines, ethanol, butyloctyl salicylate, an acrylate polymer, polyethylene glycol (PEG), propylene glycol, stearate arachidoyl alcohol, stearyl alcohol, an acrylate, xanthan gum, disodium ethylenediaminetetraacetic acid (EDTA), a retinoid, an alpha-hydroxy acid such as glycolic acid, lactic, tartaric acid, citric acid, a beta-hydroxy acid, hydroquinone, kojic acid, L-ascorbic acid, vitamin C0, a glycosaminoglycan, a copper peptide, alpha lipoic acid, dimethylaminoethanol, a hormone, a growth factor, a surfactant, a thickening agent such as hydroxyethylcellulose, xanthan, carrageenan, alginate or chemically modified cellulose compounds, an emulsifier such as isopropyl myristate (IPM) or diisopropyl adipate, or a polyacrylate, an aromatic agent such as anise alcohol, benzyl alcohol, coumarin, eugenol, hydroxycitronellal, limonene or geraniol, a polyhydroxy acid, a preservative, an antioxidant, and/or a pigment.

3. The ultraviolet light blocking composition of claim 1, wherein the bacteriophage is purified from a bacteria cell selected from E. coli or P. aeruginosa, or a cocktail of E. coli and P. aeruginosa.

4. (canceled)

5. The ultraviolet light blocking composition of claim 1, wherein the bacteriophage is a T6 phage, Pf phage, T4P phage, Qhix phage, Eb M3 phage, Luz19 phage, Luz24 phage, or a combination of two or more of the foregoing bacteriophages.

6-9. (canceled)

10. The ultraviolet light blocking composition of claim 1, wherein the secondary agent comprises a thickening agent, an emulsifier, an aromatic agent, an alpha-hydroxy acid, a polyhydroxy acid, a preservative, an antioxidant, a pigment or a combination of two or more of the foregoing, and wherein the ultraviolet light blocking composition is formulated to be a sunscreen.

11. The ultraviolet light blocking composition of claim 1, comprising a paint, an industrial coating, a stain, or a lacquer.

12. The ultraviolet light blocking composition of claim 1, comprising a solution for agricultural application to sun-sensitive crops.

13-17. (canceled)

18. A method to reduce or eliminate ultraviolet light impact on a mammal's skin surface exposed to ultraviolet light comprising coating the mammal's skin surface with a composition comprising an active agent and a secondary agent, the active agent is a bacteriophage, a viral particle, or a phage-derived nanoparticle.

19. The method of claim 18, wherein the bacteriophage, if present, is selected from the group consisting of Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales, Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and Spiraviridae,
wherein the viral particle, if present, is produced by a mycophage or a eukaryotic cell selected from the group consisting of one or more of: Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridea, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuviridae, Phycodnaviridae, Picobirnaviridae, Picornaviridae, Plamaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinvirviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, and Zobellviridae, and
wherein the phase-derived nanoparticle, if present, is an icosahedron-shaped or filamentous phage-derived nanoparticle.

20. The method of claim 18, wherein the bacteriophage is purified from a bacteria cell selected from E. coli or P. aeruginosa.

21. (canceled)

22. The method of claim 18, wherein the bacteriophage is a Pf phage, Eb M3 phage, T4P phage, T6 phage, Qhix phage, Luz19 phage, or Luz24 phage.

23. (canceled)

24. (canceled)

25. The method of claim 18, wherein the icosahedron-shaped or filamentous phage-derived nanoparticle is comprised of a phage membrane protein, a peptidoglycan, a teichoic acid, a lipid-containing phage envelope, a lipid containing phage shell, or a combination thereof.

26. The method of claim 18, wherein the secondary agent comprises one or more of glycerin, a betaines, ethanol, butyloctyl salicylate, an acrylate polymer, polyethylene glycol (PEG), propylene glycol, stearate, arachidoyl alcohol, stearyl alcohol, an acrylate, xanthan gum, disodium ethylenediaminetetraacetic acid (EDTA), a retinoid, an alpha-hydroxy acid such as glycolic acid, lactic, tartaric acid, citric acid, a beta-hydroxy acid, hydroquinone, kojic acid, L-ascorbic acid, vitamin C0, a glycosaminoglycan, a copper peptide, alpha-lipoic acid, dimethylaminoethanol, a hormone, a growth factor, a surfactant, a thickening agent such as hydroxyethylcellulose, xanthan, carrageenan, alginate or chemically modified cellulose compounds, an emulsifier such as isopropyl myristate (IPM) or diisopropyl adipate, or a polyacrylate, an aromatic agent such as anise alcohol, benzyl alcohol, coumarin, eugenol, hydroxycitronellal, limonene or geraniol, a polyhydroxy acid, a preservative, an antioxidant, and/or a pigment, and wherein composition is formulated as a sunscreen.

27-36. (canceled)

37. The methods of claim 18, wherein the composition is formulated as a paint, a stain, a lacquer, or an industrial coating.

38-46. (canceled)

47. The method of claim 18, wherein the composition is formulated as a solution for agricultural application to sun-sensitive crops.

48-51. (canceled)

52. A method to manufacture a composition for reducing or eliminating ultraviolet light impact, the method comprises combining an active agent, the active agent being bacteriophage, a viral particle, or a phage-derived nanoparticle, with a secondary agent and/or other agents.

53. The method of claim 52, wherein the bacteriophage, if present, is selected from the group consisting of Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae, Schitoviridae, Zobellviridae, Halopanivirales, Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Durnavirales, Picobirnaviridae, Ampullaviridae, Autolykiviridae, Bicaudaviridae, Globuloviridae, Guttaviridae, Halspiviridae, Plasmaviridae, Portogloboviridae, Thaspiviridae, and Spiraviridae,
wherein the viral particle, if present, is produced by a mycophage or a eukaryotic cell selected from the group consisting of one or more of: Abyssoviridae, Ackermannviridae, Adenoviridae, Adintoviridae, Aliusviridae, Alloherpesviridae, Alphaflexiviridae, Alphasatellitidae, Aplhatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridaer, Partitiviridae, Parvoviridae, Paulinoviridae, Peribunyaviridae, Permutotetraviridae, Phasmaviridae, Phenuiviridae, Phycodnaviridae, Picobirnaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymnoviridae, Virgaviridae, Wupedeviridae, Xinmoviridae, Yueviridae, an Zobellviridae,
wherein the phage-derived nanoparticle, if present, is an icosahedron-shaped or filamentous phage-derived nanoparticle, and
wherein the secondary agent comprises one or more of glycerin, a betaines, ethanol, butyloctyl salicylate, an acrylate polymer, polyethylene glycol (PEG), propylene glycol, stearate, arachidoyl alcohol, earyl alcohol, an acrylate, xanthan gum, disodium ethylenediaminetetraacetic acid (EDTA), a retinoid, an alpha-hydroxy acid such as glycolic acid, lactic, tartaric acid, beta-hydroxy acid, hydroquinone, kojic acid, L-ascorbic acid, vitamin C0, a glycosaminoglycan, a copper peptide, alpha-lipoic acid, dimethylaminoethanol, a hormone, a growth factor, a surfactant, a thickening agent such as hydroxyethylcellulose, xanthan, carrageenan, alginate or chemically modified cellulose compounds, and emulsifier such as isopropyl myristate (IPM) or diisopropyl adipate, or a polyacrylate, an aromatic agent such as anise alcohol, benzyl alcohol, coumarin, eygenol, hydroxycitronelial, limonene or geraniol, a polyhydroxy acid, a preservative, an antioxidant, and/or a pigment.

54. (canceled)

55. (canceled)

56. The method of claim 52, wherein the bacteriophage is a Pf phage, Eb M3 phage, T4P phage, T6 phage, Qhix phage, Luz19 phage, or Luz24 phage.

57. (canceled)

58. (canceled)

59. The method of claim 5, wherein the icosahedron-shaped or filamentous phage-derived nanoparticle is comprised of a phage membrane protein, a peptidoglycan, a teichoic acid, a lipid-containing phage envelope, a lipid containing phage shell, or a combination thereof.

60. (canceled)

61. The method of claim 52, wherein the composition is a sunscreen, a paint, an industrial coating, a stain, a lacquer, or a solution for agricultural application to sun-sensitive crops.

62-67. (canceled)
